(19) United States
(12) Patent Application Publication
(10) Pub. No.: US 2005/0221367 A1
(43) Pub. Date: Oct. 6, 2005

(54) Temperature gradient nucleic acid hybridization method

(76) Inventor: Nathaniel Tue Tran, Irvine, CA (US)

Correspondence Address: NATHANIEL TUE TRAN, 3205 ASPEN GROVE, IRVINE, CA, 92618, US

(21) Appl. No.: 11/098,916

(22) Filed: Apr. 4, 2005

Related U.S. Application Data
(60) Provisional application No. 60/559,685, filed on Apr. 2, 2004.

(51) Int. Cl. C12Q 1/68

(52) U.S. Cl. 435/6

(57) ABSTRACT

A novel method of nucleic acid hybridization placing immobilized nucleic acid at the lower end of a temperature gradient to achieve more efficient and more completed hybridization. Immobilized nucleic acids such as DNA array or cross-linked membrane for Northern blotting is anchored on a surface with a heat sink, while within the same hybridization chamber a heat source is place the furthest possible distance away from the array or membrane. The invention also teaches different ways to construct such a hybridization chamber and additional optional improvement features.

### CROSS REFERENCE TO RELATED APPLICATION

This application claims priority of provisional application U.S. Ser. No. 60/559685 filed Apr. 2, 2004 titled Temperature gradient DNA hybridization method. The content of this provisional application is incorporated herein as reference.

### BACKGROUND OF THE INVENTION

DNA naturally pairs into anti-parallel double strands so that A pairs with T while C pairs with G. This base-paring is commonly known as Watson-Crick base-paring. Double-stranded DNA denatures at high temperature into single strands and then renatures when the temperature is lowered below their “melting” temperature. The same principle applies to other nucleic acid such as RNA. This principle is used in many DNA and RNA techniques such as Southern blotting, Northern blotting, and DNA array hybridization to detect and quantify specific nucleic acid sequences.

DNA microarray technology has emerged as a powerful tool for discovering genetic information. The application of this revolutionary technology, embodied in what are known as DNA chips, has resulted in explosive discoveries in the fields of health-related sciences and medicine. The major applications of DNA microarrays are divided into two categories: studies of genomic structure and studies of gene expression. The former studies the present or absence of a specific sequence comprising genetic disease diagnosis (e.g., mutation detection), polymorphism analysis (e.g., SNP analysis), gene mapping, and sequencing by hybridization. The latter mainly provides information about which genes are currently active in a given sample and at what level. Such information is the basis for understanding molecular changes in diseases or drug treatments which aids in discovery of disease mechanisms and drug targets for diagnostics and therapeutics development.

In its most basic form, a DNA microarray is simply a solid support, e.g. glass or silicon, bearing on its surface an array of different DNA fragments or other nucleic acid binding agents (called “probes” or “aptamers”), usually having a known sequence, at discrete locations or spots on the support. The DNA spots on the chip are hybridized to detectably labeled nucleic acid molecules (called “targets”) which are present in a test sample. The pattern and extent of detectable label, e.g. fluorescence, that is observed provides information about the nucleic acids present in the solution, either qualitatively in searching for the presence of a particular sequence (for example, mutation detection), or quantitatively, in attempting to determine the amount of numerous sequences likely to be present (as in gene expression patterns).

Micro hybridization arrays on glass slides enable heterogeneous hybridization between the target nucleic acids and the probes. Each microarray consists of several hundred to several hundred thousand microscopic spots. Each spot in the array contains identical, single strand oligonucleotide probes which are usually 10-100 bases long or complementary DNA (cDNA) probes, typically 500-1,000 bases long. The amount of the probe attached to the solid support is small and the spots are closely spaced. Thus, the consumption of probe solution to make spots and the volume of target-containing test solution are both low. The probes are attached to the solid support by chemical linkage or chemisorption. A solution phase of DNA labeled with a detectable reporter is then poured onto the support surface. Only two complementary strands, one in the liquid phase and the other on the solid phase, will hybridize under appropriate conditions of hybridization and washing. The support is then brought to a suitable detection instrument to determine the degree of hybridization.

DNA microarray technology has many advantages in comparison to previous methods such as Southern blotting. First, microarrays enable performing analyses in parallel. Arrays consist of a large variety of different DNA spots, and a corresponding number of targets can be tested for simultaneously. Second, microarrays use very little material. Since microarrays are compact, only a small amount of biological sample is consumed, thereby reducing the cost substantially. Third, microarrays require only a limited investment for labor. Most parts of the process for generating DNA microarrays are automated and high-throughput in nature, reducing human involvement. Fourth, some microarrays are standardized and available commercially thus enable many researchers to repeat the same type of experiments with no added variation.

One of the main differences between DNA microarrays and Southern blotting that influences the hybridization process is in the use of an impermeable, solid substrate, usually glass, instead of the membrane support used in Southern blotting. Additionally, the positions of the probes and targets are reversed, i.e., in Southern blotting, the targets are disposed on the support, and the probes are in solution. The solid glass support has a number of advantages over porous membranes used in Southern blotting. The main advantage is that target molecules cannot penetrate the surface. Therefore, target nucleic acid molecules have immediate access to the probes once they contact the glass surface. In addition, the washing step following the spotting or hybridization step for removing unbound probes or unhybridized targets is also unimpeded, thereby improving hybridization reproducibility.

DNA array can be used for quantitative comparison of mRNA expression between two sources. First, the mRNA is amplified into cDNA by RT-PCT to increase the amount of targets to be detected and observed. To make the new cDNA detectable, amplification reaction usually incorporate nucleotides which are labeled with fluorescent dye like Cydye. Two different Cydye with different emission spectra are used so that the resulting cDNA can be mixed together and multiplexed on the same DNA microarray. The main problem with this method is that when it comes to hybridization, there are loose dsDNA where only one strand qualifies as target and the other strand will compete with probes or aptamers for this target strand. That makes quantitative analysis almost impossible. Our invention seeks to solve this problem.

Recent improvement in nucleic acid preparation added additional steps to make the reactions more quantitative than the above described method. Briefly, these additional steps aim at producing single-stranded targets only so competition with complementary strand is not a problem. For instance, during the RT-PCR reaction, additional sequences such as T7 polymerase promoter sequences are introduced into the cDNA (as engineered into the primers). Once the reaction yields cDNA, this cDNA can be used to produce cRNA using T7 RNA polymerase. Afterward, RNAse-free DNAse is added to digest away the entire DNA without affecting the new cRNA. This single-stranded cRNA is then used to react with DNA microarray without any competing complementary strand. While this method solves the problem, it adds another step and produces fragile RNA molecules. The extra step introduces additional variation, while RNA needs to be handled with care to avoid degradation. Our invention can eliminate the extra step and still enable quantitative analysis using the more stable dsDNA.

### SUMMARY OF THE INVENTION

The invention teaches a new method of hybridizing dsDNA or dsRNA to immobilized ssDNA or ssRNA within a temperature gradient so that the hybridization reaction becomes much more efficient enabling better quantitative analysis. The temperature gradient is set up so that the immobilized nucleic acids are at the lower temperature end of the gradient where already hybridized nucleic acids are less likely to become denatured or “unhybridized”. However, if the target nucleic acid renatures or hybridizes with its loose complementary sequences, then the renatured molecule can move toward the higher temperature end of the gradient and be denatured again to start the cycle over. Overtime, the hybridization reaction is driven toward completion by having more and more target nucleic acids hybridized to immobilized probes or aptamers.

A further improvement of the invention incorporates the variation of temperature over a period of time so that nucleic acid with different GC percentages can hybridize efficiently. This is accomplished by starting the reaction at high average temperature and then gradually reduces the average temperature as time progress. With this temperature change, a temperature gradient is still maintained during hybridization reaction. So while the average temperature begins high and then drop gradually, there are still a higher temperature end and a lower temperature end.

An additional improvement is a stirring mechanism added to the hybridization chamber to circulate the hybridization fluid mixture. To make possible the temperature gradient, one may need to increase the volume of hybridization solution by many folds. The stirring actions compensate for this dilution and enable better contact and hybridization.

An object of the invention is to teach a new hybridization method for nucleic acid especially for use with DNA array.

Another object of the invention is to teach one how to build an instrument suitable to perform such analysis.

A further object of the invention is to incorporate additional improvement such as gradual decrease in average temperature to accommodate efficient hybridization of poly nucleotide fragments with different melting temperature.

### DETAIL DESCRIPTION OF THE INVENTION

Definitions:

The term “hybridization” as used herein refers to the process of forming a duplex between two members of specific binding pair. The specific binding pair is frequently complementary or partially complementary strands of a polynucleotide. It will be understood by those skilled in the art of molecular biology that the term “polynucleotide” as used herein includes analogs of naturally occurring polynucleotides and does not covey any limitation of the length of the polynucleotide. One of the polynucleotide strands may be immobilized on a solid substrate and use to detect and quantify the other strand by hybridization.

The term denature (denaturation), melt (melting) all refer to the opposite process of hybridization where a duplex polynucleotide becomes two single strand polynucleotides usually due to high temperature, high chaotropic salt or the combination of both. Renature (renaturing) or anneal (annealing) means reverting back to the duplex form including hybridizing with the original complementary strand, hybridizing with an equivalent complementary strand or hybridizing with the immobilized aptamers.

Inventive Steps:

Quantitative comparison of expressed mRNA is made possible by first purifying and amplifying into cDNA by RT-PCR. The polymerase chain reaction (PCR) part of this amplification can be controlled so that the amplification can be arithmetic or exponential. This process is known to those skilled in the art by manipulating the availability of one primer used in the amplification process. Depending on the need of the researcher to compare gene expression, either arithmetic or exponential amplification can be used.

To enable detection and quantification, labeled nucleotides (substrate for the amplification reaction) are added to the reaction mixture. These are nucleotides that contain labels such as fluorescent dyes, biotin, radioactive isotopes, and a whole range of other markers known to those skilled in the art to enable rapid detection and quantification of labeled target nucleotides. The labels can also be part of the primers used to amplify the RNA. Such primers can be commercially synthesized by many nucleic acid companies and is usually a preferred method because using labeled primer, the size of the target nucleic acids don't affect the amount of signal thus enable comparison between genes within a sample as well as between samples.

When fluorescent dyes are used to label nucleic acids, there are several colors available to label more than one sample and then combine them for simultaneous analysis on the same array. Typical practice compares two to three samples per array. The typical dyes used are available commercially such as Cy2, Cy3, Cy5 dye set form Amersham Biosciences or Alexa fluor dyes from Molecular Probes.

Using two samples on the same array eliminate many variables such as array to array variations that can be introduced by human or machine through the process. The different mixtures of the two dyes generate different colors that can be read and analyzed by the appropriate instrumentation and computer program. The goal for most analysis is either qualitative all or none detection or quantitative comparison of more or less and by how much.

This invention teaches a novel method of hybridizing DNA to its immobilized complementary sequences such as DNA array using a temperature gradient to maximize capturing efficiency. The immobilized nucleic acid such as a DNA array is placed at the lower temperature within a temperature gradient, while the hybridizing solution can move freely toward the array or toward a higher temperature environment away from the array. The temperatures at both low and high ends are precisely controlled and are adjustable by any user. At the right temperature setting, double-stranded DNA is more likely to denature away from the DNA array and renature or anneal when they are in closed proximity to the array. Loosed ssDNA molecules that hybridize to their complementary aptamers on the array will likely to remain there because of the lower temperature, while DNA that rehybridizes to become loosed dsDNA can still move toward the hotter end by thermal agitation or active stirring/pumping and be denatured. This hybridization condition provides a favorable environment to maximize hybridization to the array.

In addition to a thermal gradient with increase temperature away from the array, a thermal gradient across the array will further increase its versatility. A DNA array can be designed with groups of higher percentage of GC (high GC content) toward one side. Upon hybridization, it is more advantageous to have higher temperature on the high-GC-content side to facilitate better hybridization. However, depending on the needs of the analysts, most arrays can be designed with a known percentage of GC content in chosen aptamers so that the same hybridization temperature can be used for the entire array.

A further improvement of this invention encompasses a programmable hybridization chamber that start out with high temperature to denature dsDNA. Then a temperature gradient is form with the DNA array at the lower temperature end. The average temperature is then lowered gradually while still maintaining the temperature gradient. DNA with high GC content will hybridize first and the rest will hybridize later. This will also serve as a universal method to automatically hybridize any nucleic samples to any arrays without knowing more information about the samples or the arrays.

Design of the Hybridization Chamber with a Temperature Gradient:

A simple design of the hybridization chamber comprises of two temperature control units, one for each side of the chamber. The array will be placed on the side that will be set as lower temperature during the hybridization process. The temperature control can be by an external heating cooling bath with separate temperature control units or as simple as a direct heating element and a cooling element. This hybridization chamber can provide temperature gradient both ways by having either side as the higher temperature side.

A simpler hybridization chamber comprises a heating element to heat one side of the chamber and a cooling element to cool the other side of the chamber. Heating elements can be as simple as a heating coil controlled by voltage or current or more complex forms known to those skilled in the art. Cooling element can be as simple as a heat-sink with or without a fan, or more complicated device such as Peltier-junction chip or other refrigerated devices known to those skilled in the art. The cooling side of the chamber further comprises means to affix or attach the array or membrane used for analysis. An optional agitation apparatus such as magnetic coupled stirring can also be added to stir the liquid within the chamber when necessary.

The heater and cooler are temperature controlled and can be adjusted by the operator within certain limits. The heater and cooler can be large radiator type heating and cooling bath with fluid running through the system as conductor. Alternatively, they can be a heating element as heater and Peltier junction device as cooler. Such a chamber can be temperature-controlled by the user directly or through programmable electronics. For instant, when the DNA sample is introduced, the entire chamber can be set to denature DNA where the entire sample gets heated. Then, after sufficient denaturing time, temperature is adjusted to create a gradient that is best for selective hybridization. This is accomplished by activating the cooler and adjusting the heater to create the desirable temperature gradient. If there is a stirrer within the system, then the stirring rate will also need to be adjusted to maintain certain temperature gradient integrity. After hybridization is completed, then temperature is adjusted to uniform temperature desirable for completing hybridization and washing off non-specific bindings.

Alternative Designs for the Temperature Gradient Hybridization Chamber:

An alternative design comprises two chambers connected by a conduit. One chamber is maintained at a higher temperature while the other is at lower temperature. Fluid is moved between the two chambers preferably through at least two conduits (one way movement); however one conduit and two way movement would also work. A pumping mechanism such as a magnetic stir bar shaped as a fan propeller is placed in a path designed for it that enables pumping. This design is known to those skilled in the art and can be learnt from observing existing fan or blower design. While magnetic coupling is preferred, other means of getting the “fan blade” to move is also acceptable including direct coupling to a motor. Many other methods of making the fluid to circulate from one chamber to the other chamber is also possible and are know to those skilled in the art.

Another alternative design uses the conduit to heat the fluid as it is passing through. This design comprises a chamber and a circulating conduit where fluid can move from this chamber through the conduit and then come back to the same chamber. A means of propelling fluid is also used to maintain this circulation. The conduit is heated while the chamber is cooled to create the necessary temperature gradient. Preferably, the fluid is taken at a point close to the array (where its temperature is at the lowest) to pass through the conduit and then return to the furthest point away from the array. The remaining fluidic movement can help dissipate the fluid

Hybridization for a membrane type macroarray of for Northern blotting can be done by a modified version of the rolling bottles. Most membrane hybridization are carried out in a bottle that rotate around so the hybridization solution is constantly moving to allow even distribution and exposure across the membrane. One way to achieve a temperature gradient in these types of bottles is to have a heating element at the center of the bottle. The bottle can be hollow to allow insertion of such a heating element. Additionally, the heating element will also serve as anchor to rotate the bottle. Temperature is control in such a way that the heating core provide the additional heat to create the warmer side of the temperature gradient while the bottle is enclose in an incubator or water bath where the air or water cool the outer part of the bottle to create the cooler side of the gradient. The adhesion of the membrane to the outer part of the bottle is usually sufficient to keep the membrane on the cooler side of the temperature gradient; however, anchors for the membrane can be added as needed.

Advanced Design:

To minimize the amount of fluid used, a chamber can be designed with a conical shape or similar where the sharp point is the higher end to the temperature gradient. To take advantage of thermal convection and enable user friendliness, this conical shape chamber is inverted so that the array can be mounted on the cover facing downward. Additional space around and above the array allows over filling of fluid so that the array can be submerged at all time. Added features that rock the array back and forth can be added to further improve performance. Alternatively the entire chamber or just the array can be vibrated while the array is placed at a slight slant to the horizontal so that air bubbles trapped by the array can be removed. Stirring is optional using a magnetic stir bar or equivalent placed inside the chamber and moved by an external dynamic magnetic field.

Supporting Methodology

The temperature gradient hybridization method only works well with arrays or membranes constructed with single strand nucleic acid as aptamers. These aptamer can be synthetic oligo nucleotides, or those created using M13 phage expression or equivalence to make ssDNA. Another method that can be used to construct ssDNA is to methylate DNA and then amplify it by PCR for one cycle, then use the resulting DNA to spot the array. Before this array is ready for use, one need to digest it with a methylation specific nuclease then denature and wash away all the remnant of the methylated DNA strand. This method can actually introduce both plus and minus strands of DNA onto the same spot of a DNA array that can randomly provide a balance to capture both strands of the target DNA. Alternatively, normal DNA after undergoing PCR can be amplified for one more cycle using newly added methylated or modified nucleotides and primers. The resulting DNA has one methylated strand and one unmethylated strand can be made into arrays; when the analyst need to use the array he can subject these arrays to enzymatic digestion to digest away the methylated strand of DNA prior to using the array for analysis. DNA can be methylated by many means that are known to those skilled in the art including enzymatic methylation using DNA methylase and methyl donor such as S-Adenosyl-Methionine (SAM), or by chemical means such as exposing to methyl bromides . . . etc. Methylated nucleotide substrates and other modified nucleotides are now available commercially.

Care must be taken that DNA originated from bacterial or mammalian sources are normally methylated, thus they should be methylated first and then amplified for one cycle. On the contrary, DNA amplified by PCR reactions are totally unmethylated, thus they can be amplified for one more cycle with modified substrate to obtain modified-unmodified hybrid dsDNA if that is desirable.

Other forms of modifications to DNA that enable an enzyme to selectively digest one strand but not the other are also possible and can be devised by those skilled in the art. Methylation is the only chosen form described here because it is the most common and is readily available.

Alternative to DNA, RNA can also be used as in Northern blot and RNA slot blot. The traditional Northern blotting use RNA immobilized on a membrane and then probes to look for the gene of interest. The probes used are normally from dsDNA sources such as PCR reactions. Using temperature gradient to perform membrane hybridization will improve the efficiency of these blots by favoring the right probes binding to immobilized RNA target sequences over similar complementary DNA sequences floating in solution.

1. A method of hybridizing loosed nucleic acids to immobilized nucleic acid comprising the steps of:
(a) providing a chamber wherein hybridization fluid is contained; and
(b) producing a temperature gradient within said chamber.

2. The method of claim 1 wherein within said chamber temperature increases with increased distance from the immobilized nucleic acids.

3. The method of claim 1 wherein said chamber further comprises:
a means for agitating said hybridization fluid.

4. The method of claim 1 further comprises:
a means for removing air bubbles accumulating on the surface supporting single strand nucleic acids.

5. The method of claim 1 further comprising a step of:
reducing the average temperature of said chamber gradually whereby different nucleic acid with different annealing temperature can hybridize.

6. A nucleic acid hybridization chamber comprising:
(a) a heating element;
(b) a cooling element; and
(c) a means to control said heating element and said cooling element to create a temperature gradient within said hybridization chamber.

7. The nucleic acid hybridization chamber of claim 6 wherein
(a) the temperature can be lowered for immobilized nucleic acid; and
(b) the temperature can be raised for a portion of hybridization solution.

8. The nucleic acid hybridization chamber of claim 6 further comprising:
a means to agitate fluid within said chamber.

9. The nucleic acid hybridization chamber of claim 6 further comprising:
a means to lower the average temperature within said chamber gradually.

10. The nucleic acid hybridization chamber of claim 6 further comprising a circulating conduit connected to said hybridization chamber.

11. The nucleic acid hybridization chamber of claim 10 wherein said heating element is used to heat said conduit.

12. The nucleic acid hybridization chamber of claim 11 further comprising a means of pumping hybridization solution through said conduit whereby fluid is circulated through said conduit and back to said hybridization chamber.

13. A method of producing single strand DNA array comprising the steps of:
(a) producing double strand DNA
(b) methylating said double strand DNA
(c) amplifying the methylated double strand DNA for one cycle by polymerase chain reaction;
(d) immobilizing the resulting amplified DNA on an array; and
(e) digesting away one strand of DNA using methylation selective enzyme.
